(19) United States
(12) Patent Application Publication
(10) Pub. No.: US 2012/0010507 A1
(43) Pub. Date: Jan. 12, 2012

(54) ULTRASOUND TRANSDUCER ARCHITECTURE HAVING NON-TRANSITORY LOCAL MEMORY STORAGE MEDIUM FOR STORING 2D AND OR 3D/4D IMAGE DATA

(75) Inventor: Chris SANDERS, REDMOND, WA (US)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 12/830,714

(22) Filed: Jul. 6, 2010

(51) Int. Cl. A61B 8/14; A61B 8/00

(52) U.S. Cl. 600/443; 600/459

(57) ABSTRACT

The embodiments of the probe additionally include at least one non-transitory local memory storage unit or medium for storing 2D and or 3D/4D imaging data that is received at the probe. The term, “non-transitory” memory storage units or medias is synonymous with “non-volatile” memory storage units or media and is used to distinguish the term from volatile electrical devices such as random access memory (RAM) or buffer memory (BF) that hold ephemeral electrical signals. Similarly, the term, “local” memory storage units or medium is used to distinguish the term, “central” memory storage units or media that are located in the processing unit to which the probe is connected. In contrast, the non-transitory local memory storage units or media are directly coupled or connected to the probe independent of the processing unit.

### FIELD

Embodiments described herein relate generally to ultrasound diagnostic imaging systems and method of fabricating the same.

### BACKGROUND

As illustrated in FIG. 1, a conventional ultrasound imaging system includes a processing unit 1, a display unit 2, a cable 3 and an ultrasound transducer unit or probe 4. The probe or transducer 4 is connected to the processing unit 1 via the cable 3. The processing unit 1 generally controls the transducer unit 4 for transmitting ultrasound pulses towards a region of interest in a patient and receiving the ultrasound echoes reflected from the patient. The processing unit 1 concurrently receives in real time the reflected ultrasound signals for further processing so as to display on the display unit 2 an image of the region of the interest.

In detail, the transducer unit 4 further includes a predetermined number of transducer elements, which are grouped into channels for transmitting and receiving the ultrasound echoes. For 2-dimensional (2D) imaging data, a number of channels ranges from 64 to 256. On the other hand, for 3-dimensional (3D) imaging data, a number of required channels often exceeds 1000's. In the above described conventional ultrasound imaging system, the transducer unit 4 concurrently is supposed to send the processing unit 1 via the cable 3 a large volume of reflected ultrasound data for real-time imaging.

Possibly, a size of the cable 3 may become physically voluminous in order to accommodate a large number of independent electrical lines. For example, for no performance loss in 3D imaging, the cable size would be as large as approximate 40 mm in diameter in order to accommodate 6000 transmitting/receiving channels if 42 American wire gauge (AWG) wires were used. Since such a thick cable is heavy and not easily manipulated, it is impractical in normal clinical settings. Since an ultrasound technician or sonographer must repeatedly move a probe over a certain area of a patient for ultrasound imaging, the heavy cable not only prevents the technician from precisely placing the probe but also probably causes repetitive injury to a sonographer over time.

For the improved handling of 3-dimensional or 4-dimensional (3D/4D) imaging data, there have been several possibilities with the use of 2-dimensional (2D) array ultrasound transducers in order to reduce the physical cable size. Although one prior attempt included multiplexing of the data before the data transmission from the probe to the processing unit, some cases resulted in poorer performance. Another prior attempt included beamforming techniques in the transducer unit, but it was difficult due to the power required to accomplish such a task. Yet another prior attempt included compression techniques which did not provide a significant reduction since the thousands of transmitting and receiving signals to and from the processing unit failed to make this approach realistic. Lastly, wireless transmission has been considered between the transducer unit and the processing unit but even if the highest transmission rate were used, it would be too slow to accommodate the voluminous 3D/4D imaging data. It remains desirable to reduce the physical cable size.

In addition to the above described concern for the cable size, it is also desired to shorten a time period of 4D ultrasound examinations in order to maximize the clinical utilization of ultrasound imaging systems. Highly skilled sonographers occupy the ultrasound system and probe and require the patient to be present for a relatively long period of time compared with some other imaging modalities. In 2D imaging, and particular 3D/4D imaging, these concerns and desires have led to an alternative approach as disclosed in the current patent application.

### BRIEF DESCRIPTION OF THE DRAWINGS

FIG. 1 is a diagram illustrating one exemplary prior art ultrasound imaging system.

FIG. 2 is a diagram illustrating a first embodiment of the probe according to the current invention.

FIG. 3 is a diagram illustrating a second embodiment of the probe according to the current invention.

FIG. 4 is a diagram illustrating a third embodiment of the probe according to the current invention.

FIG. 5 is a diagram illustrating a fourth embodiment of the probe according to the current invention.

FIG. 6A is a diagram illustrating a prospective view of one embodiment of the probe according to the current invention.

FIG. 6B is a diagram illustrating a bottom view of the above described embodiment of the probe of FIG. 6A according to the current invention.

FIG. 7A is a diagram illustrating a prospective view of one embodiment of the probe according to the current invention.

FIG. 7B is a diagram illustrating a bottom view of the above described embodiment of the probe of FIG. 7A according to the current invention.

FIG. 8 is a flow chart illustrating the steps or acts involved in one embodied process of operation in the probe according to the current invention.

FIG. 9 is a flow chart illustrating the steps or acts involved in one embodied process with respect to the operation and use of the probe according to the current invention.

FIG. 10 is a diagram illustrating one embodiment of a two-dimensional array of transducer elements in the probe according to the current invention.

FIG. 11 is a flow chart illustrating the steps or acts involved in one embodied process of operation with respect to the use of the probe according to the current invention.

### DETAILED DESCRIPTION

Embodiments of the ultrasound imaging system according to the current invention include a probe or transducer unit, a processing unit and an optional cable connecting the probe to the processing unit. In general, the embodiments of the probe include structures, components and elements of a conventional ultrasound probe. That is, one embodiment of the probe generates ultrasound pulses and transmits them towards a certain area of a patient. The embodiment also receives the ultrasound echoes reflected from the patient while many embodiments of the probe are generally hand-held devices, some are not hand-held devices.

The embodiments of the probe additionally include at least one non-transitory local memory storage unit or medium for storing 2D and or 3D/4D imaging data that is received at the probe. The term, “non-transitory” memory storage units or medias is synonymous with “non-volatile” memory storage units or media and is used to distinguish the term from volatile electrical devices such as random access memories (RAM) or buffer memories (BF) that hold ephemeral electrical signals. Similarly, the term, “local” memory storage units or media is used to refer to the probe and to distinguish the term, “central” memory storage units or media that are located in the processing unit to which the probe is operationally connected. The non-transitory local memory storage units or media store the imaging data to be used in real-time during a clinical session as the data is being collected and or subsequently used after the data collecting session.

The non-transitory local memory storage units or media are implemented in various forms and manners and are not limited to a particular group of memory devices. In this regard, one exemplary implementation of the non-transitory local memory storage units or media includes high-capacity removable storage media such as Secure Digital (SD) memory cards that are placed inside or near the probe. Another exemplary implementation of the non-transitory local memory storage units or media includes a hard disk that is fixed in the probe or placed near the probe. In any case, the non-transitory local memory storage units or media are directly coupled or connected to the probe independent of the operational connection to the processing unit.

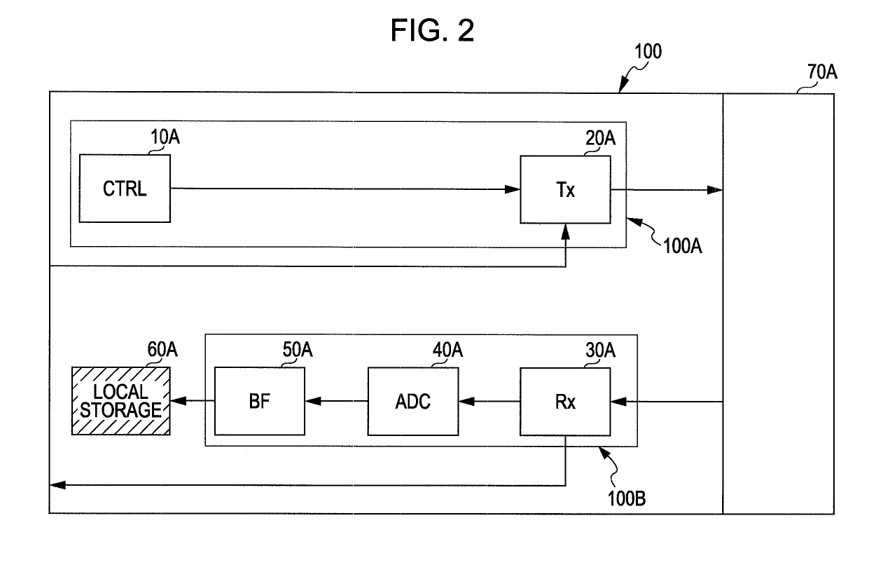

Referring now to the drawings, wherein like reference numerals designate corresponding structures throughout the views, and referring in particular to FIG. 2, a diagram illustrates a first embodiment of the probe 100 according to the current invention. In general, the first embodiment of the probe includes a transmission unit 100A, a receiving unit 100B and a transducer array unit 70A. The transmission unit 100A further includes a control unit (CTRL) 10A and a transmission circuit (Tx) 20A for controlling and producing the transmission of ultrasound pulses from the transducer array unit 70A towards a region of interest in a patient or a subject. In this regard, the transmission circuit 20A receives control information from the control unit 10A and or an external source such as a processing unit as indicated by an incoming arrow.

The receiving unit 100B further includes a receiving circuit (Rx) 30A for receiving analog signals from the transducer array unit 70A, which receives the ultrasound echoes reflected from the region of interest in the patient. The receiving circuit 30A also optionally sends out the analog signals to an external source such as a processing unit as indicated by an outgoing arrow. The receiving unit 100B also further includes an analog-to-digital convertor (ADC) 40A for converting the analog electrical signals into digital signals which are then processed by a digital beam former unit (BF) 50A. The beam former unit 50A produces beam data, and this beam data is subsequently stored in a non-transitory local memory storage or medium 60A. Although FIG. 2 does not show, the control unit 10A also outputs a signal for the handshake between the beam former unit 50A and the non-transitory local memory storage or medium 60A. In addition, a buffer memory is not shown in the diagram, the embodiment of the probe optionally requires a buffer memory in order to practice the claimed invention as well know to one of ordinary skill in the art.

The first embodiment of the probe 100 additionally includes at least one non-transitory local memory storage unit or medium 60A. Although the non-transitory local memory storage or medium 60A has a certain storage medium such as magnetic or optical characteristics to store data in a semi-permanent basis, the stored data may be erasable or rewritten over them. The non-transitory local memory storage or medium 60A also has a sufficient amount of capacity to store a predetermined amount of 2D and or 3D/4D imaging data. For illustration, the storage requirements may range from approximately 12.5 megabyte (MB) for a single one-dimensional array image to approximately 1.25 gigabytes (GB) for a ten-second video clip of two-dimensional array volume. The memory capacity of the non-transitory local memory storage or medium 60A is not limited to the above illustrated range and significantly varies depending upon various factors such as image data type, data resolution, transducer array configuration, compression techniques and so on.

In the first embodiment of the probe according to the current invention, the non-transitory local memory storage or medium 60A is located inside the probe 100. Although the diagram of FIG. 2 does not indicate, the non-transitory local memory storage or medium 60A is optionally removable from the probe 100 so that the entire stored data is transferred for further analyses in a removable medium such as a SD card after the imaging data is collected. Alternatively, although the diagram of FIG. 2 does not indicate, the non-transitory local memory storage or medium 60A such as a hard disk is optionally fixed in the probe 100 and may be accessed via a predetermined input/output port such as a universal serial bus (USB) port for transferring the stored imaging data.

In the first embodiment, the transducer array unit 70A further includes a predetermined number of transducer elements that are configured in a certain size and array for the receiving circuit 30A. For example, the transducer elements are configured in a two-dimensional array, and a certain portion such as one or more rows of the transducer elements are dedicated to receive 1D imaging data while the rest of the transducer elements is dedicated to 3D/4D imaging volume data. In this regard, the receiving circuit 30A also optionally outputs the analog 1D array imaging data to a display for confirming a region of interest based upon real-time 2D images while the receiving circuit 30A concurrently stores the corresponding 3D/4D imaging volume data in the non-transitory local memory storage or medium 60A for later reconstruction and analyses.

In the first embodiment, the above described input and output connections between the probe 100 and any external resources are any combination of wired and wireless connections.

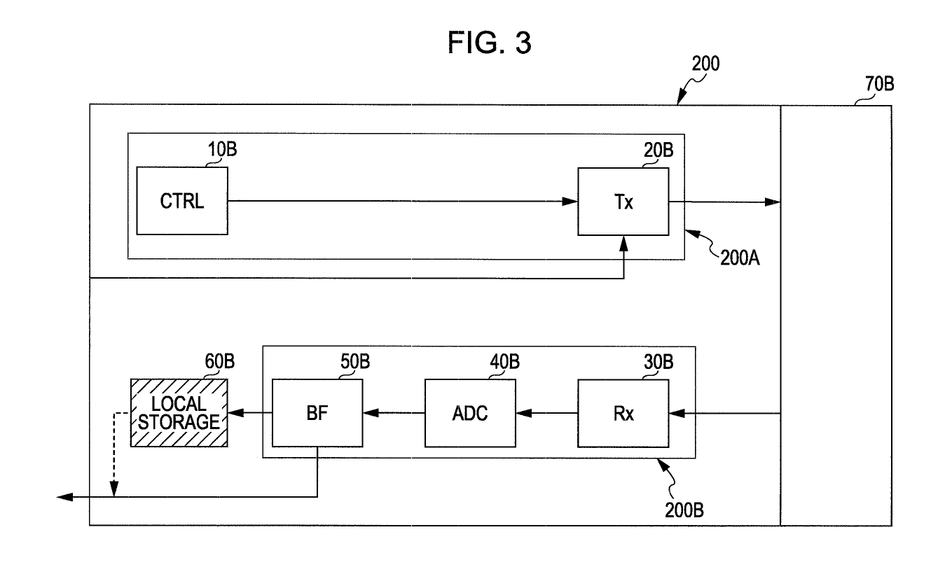

Now referring in particular to FIG. 3, a diagram illustrates a second embodiment of the probe 200 according to the current invention. In general, the second embodiment of the probe includes a transmission unit 200A, a receiving unit 200B and a transducer array unit 70B. The transmission unit 200A further includes a control unit (CTRL) 10B and a transmission circuit (Tx) 20B for controlling and producing the transmission of ultrasound pulses from the transducer array unit 70B towards a region of interest in a patient or a subject. In this regard, the transmission circuit 20B receives control information from the control unit 10B and or an external source such as a processing unit as indicated by an incoming arrow.

The receiving unit 200B further includes a receiving circuit (Rx) 30B for receiving analog signals from the transducer array unit 70B, which receives the ultrasound echoes reflected from the region of interest in the patient. The receiving unit 200B further includes an analog-to-digital convertor (ADC) 40B for converting the analog electrical signals into digital signals which are then processed by a digital beam former unit (BF) 50B. The beam former 50B produces beam data, and this beam data is subsequently stored in a non-transitory local memory storage or medium 60B. In addition, beam former unit 50B also optionally sends out the converted digital signals to an external source such as a processing unit as indicated by an outgoing arrow. Although FIG. 3 does not show, the control unit 10B also outputs a signal for the handshake between the beam former unit 50B and the non-transitory local memory storage or medium 60B. Lastly, a buffer memory is not shown in the diagram, the embodiment of the probe optionally requires a buffer memory in order to practice the claimed invention as well know to one of ordinary skill in the art.

The second embodiment of the probe 200 additionally includes at least one non-transitory local memory storage unit or medium 60B. Although the non-transitory local memory storage or medium 60B has a certain storage medium such as magnetic or optical characteristics to store data in a semi-permanent basis, the stored data may be erasable or rewritten over them. The non-transitory local memory storage or medium 60B also has a sufficient amount of capacity to store a predetermined amount of 2D and or 3D/4D imaging data. For illustration, the storage requirements may range from approximately 12.5 megabyte (MB) for a single one-dimensional array image to approximately 1.25 gigabytes (GB) for a ten-second video clip of two-dimensional array volume. The memory capacity of the non-transitory local memory storage or medium 60B is not limited to the above illustrated range and significantly varies depending upon various factors such as image data type, data resolution, transducer array configuration, compression techniques and so on.

In the second embodiment of the probe according to the current invention, the non-transitory local memory storage or medium 60B is located inside the probe 200. Although the diagram of FIG. 3 does not indicate, the non-transitory local memory storage or medium 60B is optionally removable from the probe 200 so that the entire stored data is transferred for further analyses in a removable medium such as a SD card after the imaging data is collected. Alternatively, although the diagram of FIG. 3 does not indicate, the non-transitory local memory storage or medium 60B such as a hard disk is optionally fixed in the probe 200 and may be accessed via a predetermined input/output port such as a universal serial bus (USB) port for transferring the stored imaging data.

In the second embodiment, the transducer array unit 70B can be electronically configured in several ways. For example, given a 2D array, one or more rows of elements could be electronically connected and thus configured to receive 2D data. Subsequently, the 2D array could be electronically configured to receive 3D/4D data. The 2D imaging data is optionally sent to the system for further processing and viewing. However, the transducer elements may be electronically configured as a 2D array to capture 3D/4D volumes.

In this regard, the beam former unit 50B also optionally outputs a certain portion of the converted digital signals such as 2D imaging data to a display for confirming a region of interest based upon real-time images. The array can then be electronically configured as a 2D array and can ultimately store the corresponding 3D/4D imaging data in the non-transitory local memory storage or medium 60B for later reconstruction and analyses. Furthermore, the non-transitory local memory storage or medium 60B includes an additional output option for outputting a certain portion such as 1D imaging data of the stored 3D/4D imaging volume data in combination with outputting the rest of the stored 3D/4D imaging volume data as indicated by a dotted line.

In the second embodiment, the above described input and output connections between the probe 200 and any external resources are any combination of wired and wireless connections.

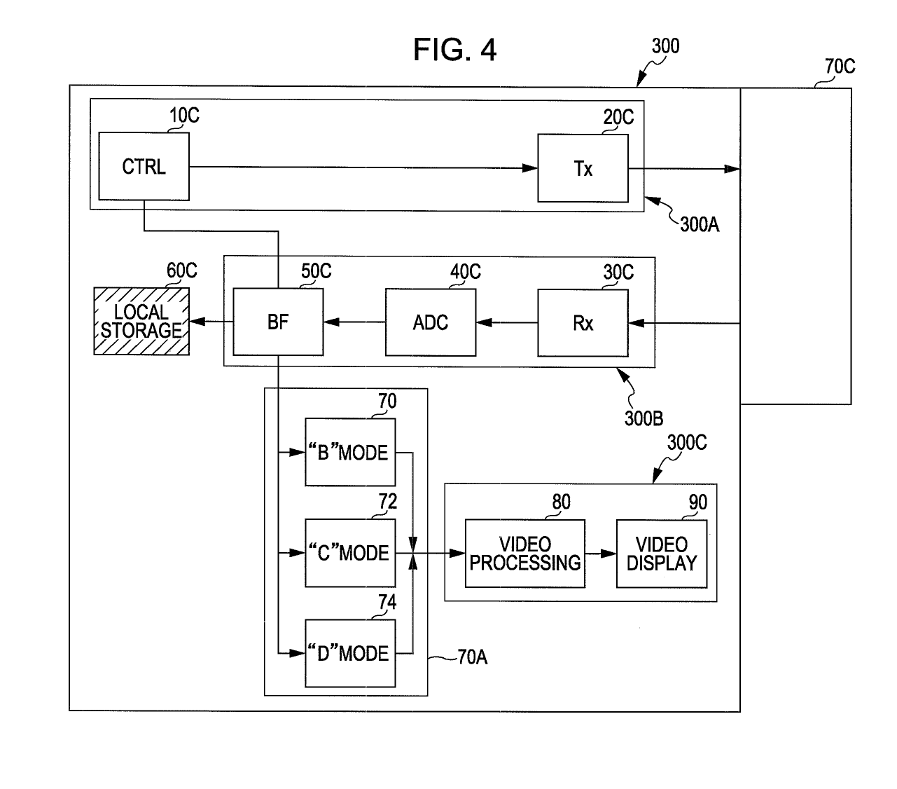

Now referring in particular to FIG. 4, a diagram illustrates a third embodiment of the probe 300 according to the current invention. In general, the third embodiment of the probe includes a transmission unit 300A, a receiving unit 300B, an on-board display unit 300C and a transducer array unit 70C. The transmission unit 200A further includes a control unit (CTRL) 10C and a transmission circuit (Tx) 20C for controlling the generation and transmission of ultrasound pulses from the transducer array unit 70C towards a region of interest in a patient or a subject. In this regard, the transmission circuit 20C receives control information from the control unit 10C as indicated by an incoming arrow.

The receiving unit 300B further includes a receiving circuit (Rx) 30C for receiving analog signals from the transducer array unit 70C, which receives the ultrasound echoes reflected from the region of interest in the patient. The receiving unit 300B further includes an analog-to-digital convertor (ADC) 40C for converting the analog electrical signals into digital signals which are then processed by a digital beam former unit (BF) 50C. The beam former 50C produces beam data, and this beam data is subsequently stored in a non-transitory local memory storage or medium 60C. Although FIG. 4 does not show, the control unit 10C also outputs a signal for the handshake between the beam former unit 50C and the non-transitory local memory storage or medium 60C. Additionally, the control unit 10C provides for control for 300B, 300C, 70, 72, 74, and 60C. Alternatively, these same units may have embedded controllers within them. Lastly, a buffer memory is not shown in the diagram, the embodiment of the probe optionally requires a buffer memory in order to practice the claimed invention as well know to one of ordinary skill in the art.

The beam former unit 50C also optionally outputs the converted digital beam data to an internal resource such as a data processing unit 70A and/or an on-board video processing unit 300C for a display as indicated by an outgoing arrow. The on-board display unit 300C further includes a video processing circuit or module 80 and a video display 90 for displaying images in response to a certain mode. The beam former unit 50C provides the digital beam data to an optional data processing unit 70A, which is further comprised of an optional “B” mode processing unit or module 70, “C” mode processing unit or module 72 and “D” mode processing unit or module 74 based upon a desired processing mode. The B mode processing unit or module 70 processes the digital beam data according to a black-and-white basic echo processing mode. The C mode processing unit or module 72 processes the digital beam data according to a processing mode to show flow with colors such as blue and red. The D mode processing unit or module 74 processes the digital beam data according to a Doppler processing mode to show time movement and flow. For example, the video processing circuit or unit 80 processes the imaging data from the C mode processing unit or module 72 and outputs to the video display 90 onboard the probe 300. The terms, module or unit are used to mean that each of these elements is optionally implemented in hardware, software or in combination of both.

The third embodiment of the probe 300 additionally includes at least one non-transitory local memory storage unit or medium 60C. Although the non-transitory local memory storage or medium 60C has a certain storage medium such as magnetic or optical characteristics to store data in a semi-permanent basis, the stored data may be erasable or rewritten over them. The non-transitory local memory storage or medium 60C also has a sufficient amount of capacity to store a predetermined amount of 2D and or 3D/4D imaging data. For illustration, the storage requirements may range from approximately 12.5 megabyte (MB) for a single one-dimensional array image to approximately 1.25 gigabytes (GB) for a ten-second video clip of two-dimensional array volume. The memory capacity of the non-transitory local memory storage or medium 60C is not limited to the above illustrated range and significantly varies depending upon various factors such as image data type, data resolution, transducer array configuration, compression techniques and so on.

In the third embodiment of the probe according to the current invention, the non-transitory local memory storage or medium 60C is located inside the probe 300. Although the diagram of FIG. 4 does not indicate, the non-transitory local memory storage or medium 60C is optionally removable from the probe 300 so that the entire stored data is transferred for further analyses in a removable medium such as a SD card after the imaging data is collected. Alternatively, although the diagram of FIG. 4 does not indicate, the non-transitory local memory storage or medium 60C such as a hard disk is optionally fixed in the probe 200 and may be accessed via a predetermined input/output port such as a universal serial bus (USB) port for transferring the stored imaging data.

In the third embodiment, the transducer array unit 70C can be electronically configured for 2D imaging or 3D/4D imaging. For example, the transducer elements can be configured in a two-dimensional array by configuring a certain portion such as a central row of the transducer elements to transmit and receive 2D imaging data. Subsequently, the array can be electronically configured to transmit and receive 3D/4D imaging volume data. In this regard, the beam former unit 50C also optionally outputs a certain portion of the converted digital signals such as 2D imaging data to data processing unit 70A and/or the board display unit 300C, for confirming a region of interest based upon real-time images. Once identified, the array can be electronically configured as a 2D array for capturing 3D/4D data. In this mode, the beam transformer can store the corresponding 3D/4D imaging data in the non-transitory local memory storage or medium 60C for later reconstruction and analyses.

In the third embodiment, although the probe 300 as illustrated in FIG. 4 does include any explicit input and output connections between the probe 300 and any external resources, such connections are optionally implemented in any combination of wired and wireless connections.

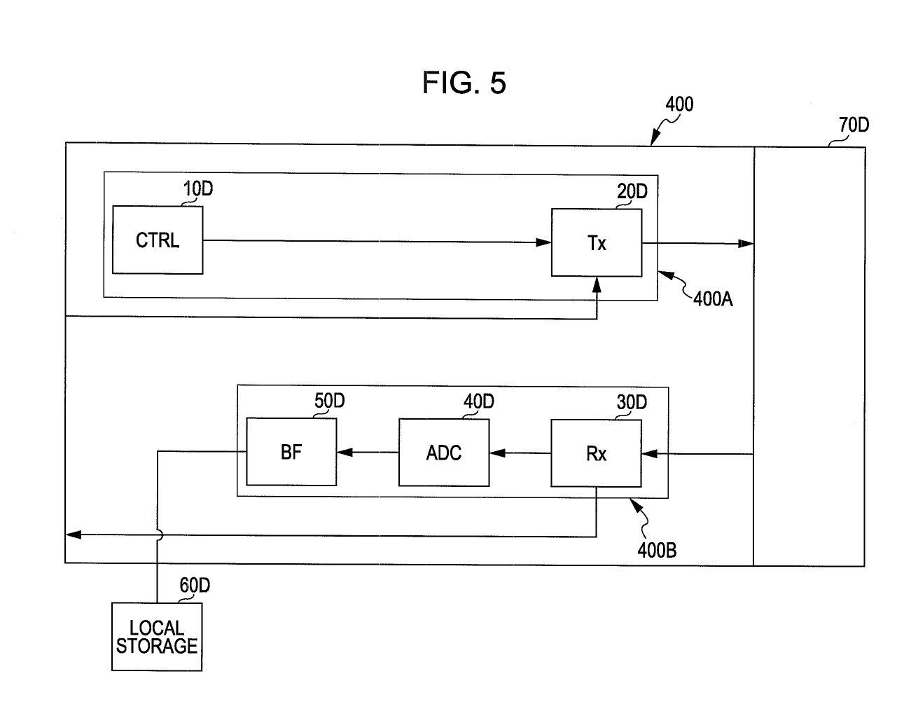

Now referring in particular to FIG. 5, a diagram illustrates a fourth embodiment of the probe 400 according to the current invention. In general, the fourth embodiment of the probe includes a transmission unit 400A, a receiving unit 400B and a transducer array unit 70D. The transmission unit 400A further includes a control unit (CTRL) 10D and a transmission circuit (Tx) 20D for controlling the generation and transmission of ultrasound pulses from the transducer array unit 70D towards a region of interest in a patient or a subject. In this regard, the transmission circuit 20D receives control information from the control unit 10D and or an external source such as a processing unit as indicated by an incoming arrow.

The receiving unit 400B further includes a receiving circuit (Rx) 30D for receiving analog signals from the transducer array unit 70D, which receives the ultrasound echoes reflected from the region of interest in the patient. The receiving circuit 30D also optionally sends out the analog signals to an external source such as a processing unit as indicated by an outgoing arrow. The receiving unit 400B also further includes an analog-to-digital convertor (ADC) 40D for converting the analog electrical signals into digital signals which are then processed by a digital beam former unit (BF) 50D. The beam former 50D produces beam data, and this beam data is subsequently stored in a non-transitory local memory storage or medium 60D. Although FIG. 5 does not show, the control unit controls modules or units within 400B, or alternatively 400B may be controlled from within i.e. embedded within sub-units. Lastly, a buffer memory is not shown in the diagram, the embodiment of the probe optionally requires a buffer memory in order to practice the claimed invention as well know to one of ordinary skill in the art.

The fourth embodiment of the probe 400 additionally includes at least one non-transitory local memory storage unit or medium 60D. Although the non-transitory local memory storage or medium 60D has a certain storage medium such as magnetic or optical characteristics to store data in a semi-permanent basis, the stored data may be erasable or rewritten over them. The non-transitory local memory storage or medium 60D also has a sufficient amount of capacity to store a predetermined amount of 2D and or 3D/4D data. For illustration, the storage requirements may range from approximately 12.5 megabyte (MB) for a single one-dimensional array image to approximately 1.25 gigabytes (GB) for a ten-second video clip of two-dimensional array volume. The memory capacity of the non-transitory local memory storage or medium 60D is not limited to the above illustrated range and significantly varies depending upon various factors such as image data type, data resolution, transducer array configuration, compression techniques and so on.

In the fourth embodiment of the probe according to the current invention, the non-transitory local memory storage or medium 60D is located outside or near the probe 400. Although the diagram of FIG. 5 does not indicate, the non-transitory local memory storage or medium 60D such as an external hard disk is placed outside or near the probe 400 and is optionally connected via a predetermined input/output port such as a universal serial bus (USB) port for transferring the stored imaging data. The above connection between the probe 400 and the non-transitory local memory storage or medium 60D is direct and independent of other connections such as to the external processing unit. Furthermore, the above connection between the probe 400 and the non-transitory local memory storage or medium 60D is optionally cabled or wireless.

In the fourth embodiment, referring to the transducer array unit 70D, given a 2D array, one or more rows of elements could be electronically connected and thus configured to receive 2D imaging data from 1 1D array. Subsequently, the 2D array could be electronically configured to receive 3D/4D imaging data as a 2D array. The receiving circuit 30D also optionally outputs the analog array imaging data to a display for confirming a region of interest based upon real-time 2D images. Subsequent to the 2D imaging activity, the receiving circuit 30D can store the corresponding 3D/4D imaging volume data in the non-transitory local memory storage or medium 60D for later reconstruction and analyses. The 1D array imaging data is optionally sent to the system for further processing and viewing. However, the transducer elements or array may be electronically configured as a 2D array to capture 3D/4D volumes.

In the fourth embodiment, the above described input and output connections between the probe 400 and any external resources are any combination of wired and wireless connections.

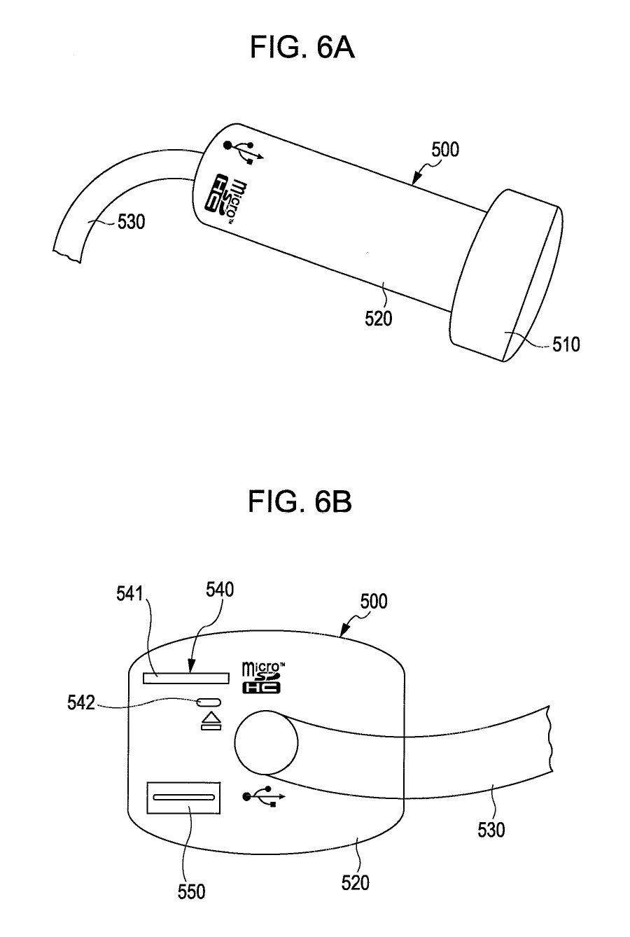

Now referring to FIG. 6A, a diagram illustrates a prospective view of one embodiment of the probe according to the current invention. The embodiment of a probe 500 includes a probe head portion 510, a probe handle portion 520 and a connection cable 530, which connects the probe 500 to a processing unit. In general, an ultrasound technician holds the probe 500 by the probe handle portion 520 in order to place the probe head portion 510 over a region of interest of a patient during a data-collecting session. The probe 500 generates ultrasound pulses and transmits them from the probe head portion 510 towards the region of interest. To generate the ultrasound pulses, the probe 500 optionally receives control information from the processing unit via the connection cable 530.

Still referring to FIG. 6A, the probe head portion 510 receives the ultrasound echoes reflected from the region of interest in the patient. The probe 500 generates 2D and or 3D/4D imaging data based upon the reflected ultrasound echoes. As FIG. 6A indicates on a surface of the probe handle portion 520, the probe 500 further includes at least one non-transitory local memory storage unit or medium such as a Micro Secure Digital High Capacity (SDHC) card for storing the 2D and or 3D/4D imaging data. The probe 500 also optionally outputs some of the generated signals to an external source such as the processing unit via the connection cable.

Now referring to FIG. 6B, a diagram illustrates a bottom view of the above described embodiment of the probe 500 according to the current invention. The embodiment of the probe 500 has a bottom surface on the probe handle portion 520, and the bottom surface includes a Micro Secure Digital High Capacity (SDHC) card access portion 540, a Micro SDHC card 541, a SDHC ejection button 542, a Universal Serial Bus (USB) port 550 and a connection cable 530, which connects the probe 500 to a processing unit. The Micro SDHC card 541 is removably inserted into the SDHC card access portion 540 to be ready for storing the imaging data. When the imaging data is stored, the Micro SDHC card 541 is ejected from the SDHC card access portion 540 by pressing the SDHC ejection button 542 so that the stored data may be transferred to another system for further analysis in the removed Micro SDHC card 541. Alternatively, the imaging data that is stored in the Micro SDHC card 541 is optionally accessed via the USB port 550 without removing the Micro SD HC card 541 from the probe 500.

The above described embodiment of the probe 500 is only illustrative and provides only one exemplary implementation. The use of a Micro SDHC card and a USB port is not necessary to practice the claimed invention, and other implementations include non-transitory local memory storage media other than the Micro SDHC card and or other input/output ports other than the USB port. In this regard, one alternative embodiment includes a non-transitory local memory storage unit such as a mini hard disk drive that is fixedly placed inside the probe 500. Yet, another alternative embodiment includes a hard disk drive that is placed outside but near the probe 500 via a direct connection to the probe 500 that is independent of the cable connection 530. The direct and independent connection is optionally wired or wireless.

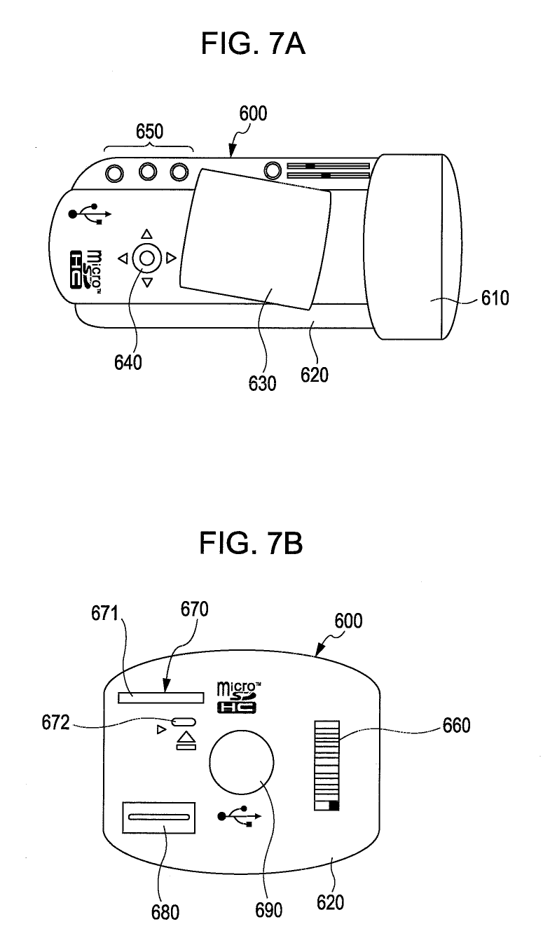

Now referring to FIG. 7A, a diagram illustrates a prospective view of one embodiment of the probe according to the current invention. The embodiment of a probe 600 includes a probe head portion 610, a probe handle portion 620, an on-board display 630, a display control 640 and operational control 650. In general, an ultrasound technician holds the probe 600 by the probe handle portion 620 in order to place the probe head portion 610 over a region of interest of a patient during a data-collecting session. The probe 600 generates ultrasound pulses and transmits them from the probe head portion 610 towards the region of interest. To control the ultrasound pulses, the user optionally inputs certain control information via the operation control buttons 650. Furthermore, the probe 600 also optionally receives some other control information from an external processing unit via wireless connection. Although the external processing unit is not illustrated in the drawing, the wireless connection is indicated by a zigzag line.

Still referring to FIG. 7A, the probe head portion 610 receives the ultrasound echoes reflected from the region of interest in the patient. The probe 600 generates 2D and or 3D/4D imaging data based upon the reflected ultrasound echoes. As FIG. 7A indicates on a surface of the probe handle portion 620, the probe 600 further includes at least one non-transitory local memory storage unit or medium such as a Micro Secure Digital High Capacity (SDHC) card for storing the 2D and or 3D/4D imaging data. The probe 600 also optionally outputs some of the generated signals to an external source such as the processing unit via the wireless connection.

In addition, the probe 600 further includes the on-board display 630 for displaying the imaging data. The displayed imaging data comes from the non-transitory local memory storage unit or medium or the currently received imaging data from the probe head portion 610. In other words, the on-board display 630 displays either real-time images or stored images as it is controlled via a display control 640. As shown by the directed triangles and circles, the display control 640 includes various display controls for manipulating a display image on the on-board display 630.

Now referring to FIG. 7B, a diagram illustrates a bottom view of the above described embodiment of the probe 600 according to the current invention. The embodiment of the probe 600 has a bottom surface on the probe handle portion 620, and the bottom surface includes a docking port 660, a Micro Secure Digital High Capacity (SDHC) card access portion 670, a Micro SDHC card 671, a SDHC ejection button 672, a Universal Serial Bus (USB) port 680 and an optional power cord 690. The handle portion 620 houses a battery, which is recharged by electricity supplied to the probe 600 via the optional power cord 690 and or the docking port 660. The Micro SDHC card 671 is removably inserted into the SDHC card access portion 670 to be ready for storing the imaging data. When the imaging data is stored, the Micro SDHC card 671 is ejected from the SDHC card access portion 670 by pressing the SDHC ejection button 672 so that the stored data may be transferred to another system for further analysis in the removed Micro SDHC card 671. Alternatively, the imaging data that is stored in the Micro SDHC card 671 is optionally accessed via the USB port 680 without removing the Micro SD HC card 671 from the probe 600.

The above described embodiment of the probe 600 is only illustrative and provides only one exemplary implementation. The use of a Micro SDHC card and a USB port is not necessary to practice the claimed invention, and other implementations include non-transitory local memory storage media other than the Micro SDHC card and or other input/output ports other than the USB port. In this regard, one alternative embodiment includes a non-transitory local memory storage unit such as a mini hard disk drive that is fixedly placed inside the probe 600. Yet, another alternative embodiment includes a hard disk drive that is placed outside but near the probe 600 via a direct connection to the probe 600 that is independent of the wireless connection. The direct and independent connection is optionally wired or wireless.

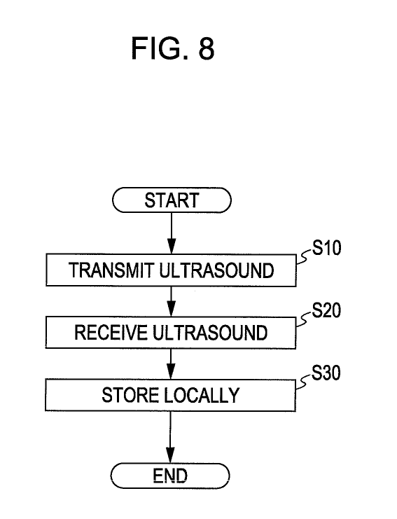

FIG. 8 is a flow chart illustrating the steps or acts involved in one embodied process of operation in the probe according to the current invention. The embodied process of operation in the probe starts in a step S10, in which ultrasound pulses are transmitted towards a region of interest and in a step S20, in which the reflected ultrasound echoes are received from the region of interest. In one embodied process, the steps S10 and S20 are repeated while the received image data is being stored in a non-transitory local storage unit or medium in a step S30. In another embodied process, the steps S10 and S20 are sequentially repeated, and then the received image data is being stored in a non-transitory local storage unit or medium in a step S30. In either embodied process, the received image signal from the step S20 is optionally further processed before storing in the step S30. One optional further process is to convert the received analog signal into digital signal and or to transfer the received analog signals to a pod or a system for additional processing and viewing.

Still referring to FIG. 8, in the storing step S 30, 2D and or 3D/4D imaging data that is received at the probe, is stored in at least one non-transitory local memory storage unit or medium. The term, “non-transitory” memory storage units or medias is synonymous with “non-volatile” memory storage units or media and is used to distinguish the term from volatile electrical devices such as random access memories (RAM) or buffer memories (BF) that hold ephemeral electrical signals. Similarly, the term, “local” memory storage units or media is used to refer to the probe and to distinguish the term, “central” memory storage units or media that are located in the processing unit to which the probe is operationally connected. The non-transitory local memory storage units or media store the imaging data to be used in real-time during a clinical session as the data is being collected and or subsequently used after the data collecting session.

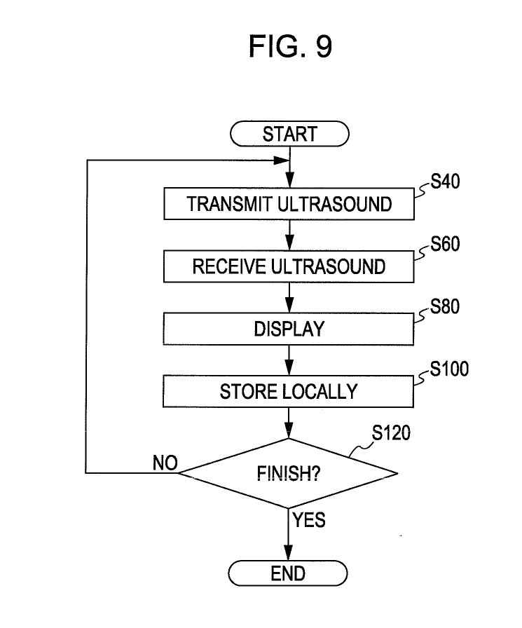

FIG. 9 is a flow chart illustrating the steps or acts involved in one embodied process with respect to the operation and use of the probe according to the current invention. The embodied process of operation in the probe starts in a step S40, in which ultrasound pulses are transmitted towards a region of interest and in a step S60, in which the reflected ultrasound echoes are received from the region of interest. In one embodied process, the steps S40 and S60 are repeated while the received image data is concurrently being displayed in a step S80 and simultaneously stored in a non-transitory local storage unit or medium in a step S100. In another embodied process, the steps S40 and S60 are sequentially repeated, and then the received image data is being displayed in the step S80 and stored in a non-transitory local storage unit or medium in the step S100. In either embodied process, the transmit step S40, the receive step S60, the display step S80 and the storing step S100 are repeated until the user terminates the process in a step S120. In addition, the received image signal from the step S60 is optionally further processed before storing in the step S100. One optional further process is to convert the received analog signal into digital signal.

Still referring to FIG. 9, in the storing step S 100, 2D and or 3D/4D imaging data that is received at the probe, is stored in at least one non-transitory local memory storage unit or medium. The term, “non-transitory” memory storage units or medias is synonymous with “non-volatile” memory storage units or media and is used to distinguish the term from volatile electrical devices such as random access memories (RAM) or buffer memories (BF) that hold ephemeral electrical signals. Similarly, the term, “local” memory storage units or media is used to refer to the probe and to distinguish the term, “central” memory storage units or media that are located in the processing unit to which the probe is operationally connected. The non-transitory local memory storage units or media store the imaging data to be used in real-time during a clinical session as the data is being collected and or subsequently used after the data collecting session.

In one embodied process, the display step S80 displays image data to the user of the probe for visually guiding the user to place the probe on a desirable position and angle before the imaging data is stored in the non-transitory local memory storage units or media. For example, the display step S80 displays real-time 2D image of the region of interest such as an organ in a patient so that the probe may be more accurately placed with respect to the organ. In this way, the user optionally initiates the storing step S100 based upon the visual confirmation of a predetermined target during the display step S80. Furthermore, also optionally in the display step S80, the user selects 2D and or 3D/4D imaging data to be stored in the non-transitory local memory storage units or media in the storing step S100.

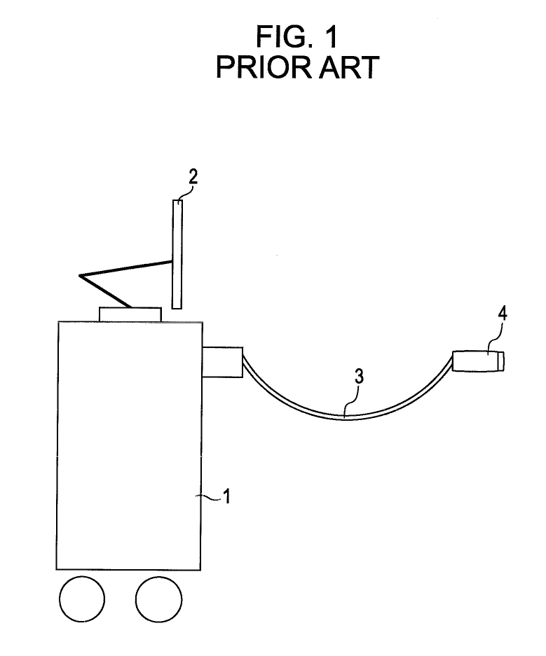

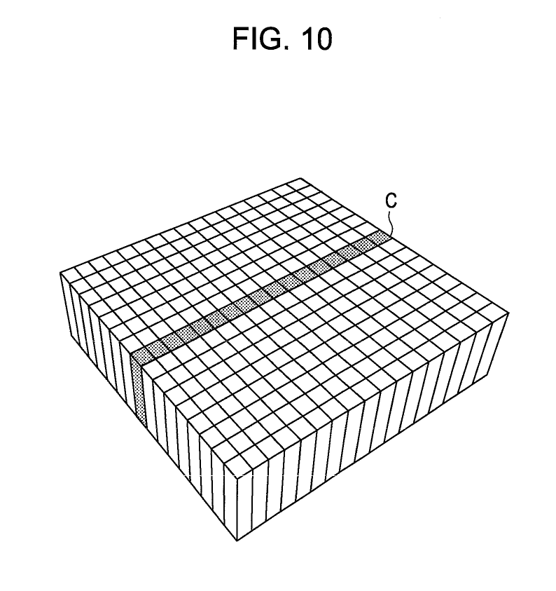

FIG. 10 is a diagram illustrating one embodiment of a two-dimensional array of transducer elements in the probe according to the current invention. The illustrative embodiment has two-dimensional array transducer elements that can be electronically configured into one row, thus behaving as a 1D array. The transducer elements of portion C are located in a one-dimensional central row and are dedicated to provide 2D image data to be displayed on a display monitor. On the other hand, the array can be electronically configured to treat all of the elements independently as a traditional 2D array. In this way, the array can provide 3D/4D image data to be initially stored in a non-transitory local storage medium or unit. As already described in one embodied process with respect to FIG. 9, a user sees real-time 2D images of a region of interest such as an organ in a patient so that a user places the probe more accurately with respect to the organ. In this way, the user optionally initiates to store 2D and or 3D/4D imaging data in a non-transitory local storage medium or unit based upon the visual confirmation of a predetermined target as he or she examines the display monitor. To implement the above process, the dedicated groups of the transducer elements are efficiently implemented in the probe according to the current invention. Furthermore, the display monitor is implemented as either a part of the processing system as illustrated in FIG. 1 or a part of the probe as illustrated in FIG. 7A.

The above described design of the transducer elements is merely illustrative and is not required to practice the claimed invention. Although the specification does not elaborate other variations, various configurations are optionally included in grouping the transducer elements and dedicating their inputs based upon the ordinary skill in the art in order to practice the claimed invention.

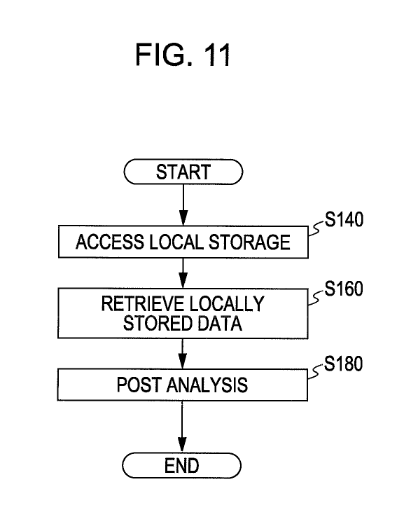

FIG. 11 is a flow chart illustrating the steps or acts involved in one embodied process of operation with respect to the use of the probe according to the current invention. The embodied process of operation with respect to the probe includes a step S140 of accessing data in a non-transitory local storage medium or unit that is associated with the probe. If the non-transitory local storage medium or unit is removably placed or fixedly placed inside the probe after storing the relevant image data, the accessing step S140 is optionally practiced by accessing the non-transitory local storage medium or unit via a predetermined input/output port on the probe. On the other hand, if the non-transitory local storage unit is placed outside and near the probe, the accessing step S140 is optionally practiced by accessing the non-transitory local storage unit via a predetermined input/output port either on the probe or the non-transitory local storage unit. Alternatively, if a non-transitory local storage medium or unit is removably placed in the probe, the non-transitory local storage medium or unit is removed from the probe in the accessing step S140. After successfully accessing the non-transitory local storage medium or unit in the step S140, relevant data is retrieved from the non-transitory local storage medium or unit in a step S160. In case of using an input/output port, the desired data is optionally transferred through the input/output port. If a non-transitory local storage medium or unit is removed from the probe in the accessing step S140, the removed non-transitory local storage medium or unit is further arranged in a device or a connection for the data retrieval step S160. Lastly, the retrieved data is further analyzed or processed in a step S180. One exemplary process is to retrieve 3D/4D data from a non-transitory local storage medium or unit of the probe after the above data is initially stored in the non-transitory local storage medium or unit of the probe. The retrieved 3D/4D data is processed to be displayed for further analyses or diagnoses independent of its data sampling session.

According to any and all embodiments explained above, advantages include an improved or efficient workflow of the ultrasound examination based upon the locally stored imaging data. Similar to other modalities such as X-ray, computer tomography (CT) or magnetic resonance imaging (MRI), the improved workflow involves a process where patients are examined using an ultrasound imaging device as quickly as possible to acquire data and the stored diagnostic images are manipulated and analyzed typically after the patients have already left the examination sessions. Particularly, in the advent of volumetric imaging or also known as sonographic tomography, a single sweep of a region using modern 2D array ultrasound may provide all necessary data for later analyses. To ensure that the transducer is positioned to capture a diagnostically relevant volume and that the system parameters are appropriately set for meaningful data, and to reduce power, a conventional 2D image is optionally provided as a good acoustic window to the operator before initiating the diagnostically relevant 3D/4D data. Ultimately, the improved workflow enhances the utilization of the imaging devices.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope of the inventions.

What is claimed is:

1. A method of ultrasound imaging, comprising the steps of:
transmitting ultrasound pulses from a probe towards a region of interest of a subject;
receiving at the probe ultrasound pulses reflected from the region of interest of the subject; and
storing data corresponding to the reflected ultrasound echoes in a non-transitory local storage medium that is directly coupled with the probe.

2. The method of ultrasound imaging according to claim 1 wherein the non-transitory local storage medium is located in the probe.

3. The method of ultrasound imaging according to claim 2 wherein the non-transitory local storage medium is removable from the probe.

4. The method of ultrasound imaging according to claim 1 wherein the probe is operationally connected to a processing unit via a control connection.

5. The method of ultrasound imaging according to claim 4 wherein the control connection is a cable.

6. The method of ultrasound imaging according to claim 4 wherein the control connection is wireless.

7. The method of ultrasound imaging according to claim 4 wherein the non-transitory local storage medium is connected to the probe independent of the control connection.

8. The method of ultrasound imaging according to claim 4 wherein the non-transitory local storage medium is located inside the probe.

9. The method of ultrasound imaging according to claim 4 wherein the non-transitory local storage medium is located outside and near the probe.

10. The method of ultrasound imaging according to claim 1 further comprising additional step of displaying 2D images based upon the reflected ultrasound echoes.

11. The method of ultrasound imaging according to claim 1 further comprising additional step of displaying 3D images based upon the reflected ultrasound echoes.

12. The method of ultrasound imaging according to claim 1 further comprising additional step of displaying 2D images based upon the reflected ultrasound echoes while said storing step stores the data for 3D/4D images based upon the reflected ultrasound echoes.

13. The method of ultrasound imaging according to claim 1 further comprising additional step of displaying 2D images based upon the reflected ultrasound echoes wherein said storing step is initiated based upon said displaying step to store the data for 3D/4D images based upon the reflected ultrasound echoes.

14. The method of ultrasound imaging according to claim 1 wherein the probe is a hand-held device.

15. An ultrasound probe, comprising:
a transmission unit for transmitting ultrasound pulses towards a region of interest of a subject;
a receiving unit for receiving ultrasound echoes reflected from the region of interest of the subject; and
a non-transitory local storage medium directly coupled to said receiving unit for storing data corresponding to the reflected ultrasound echoes.

16. The ultrasound probe according to claim 15 wherein said non-transitory local storage medium is located inside the probe.

17. The ultrasound probe according to claim 16 wherein said non-transitory local storage medium is removably placed in the probe.

18. The ultrasound probe according to claim 15 further comprising an input/output port for connecting said non-transitory local storage medium to access the data stored in said non-transitory local storage medium.

19. The ultrasound probe according to claim 15 further comprising a docking port for accessing an external power supply.

20. The ultrasound probe according to claim 15 further comprising a connection cable for connecting to an external processing unit.

21. The ultrasound probe according to claim 15 further comprising a wireless transmission unit for wirelessly connecting to an external processing unit.

22. The ultrasound probe according to claim 15 further comprising an input/output port for connecting said receiving unit to access the data.

23. The ultrasound probe according to claim 15 further comprising:
an onboard processing unit for processing the data; and
a displaying unit located on the probe and connected to said onboard processing unit for displaying the data.

24. The ultrasound probe according to claim 15 wherein the probe is a hand-held device including a battery.

25. An ultrasound imaging system, comprising:
an ultrasound probe further comprising:
a transmission unit for transmitting ultrasound pulses towards a region of interest of a subject, and
a receiving unit for receiving ultrasound echoes reflected from the region of interest of the subject;
a non-transitory local storage medium directly coupled to said receiving unit for storing data corresponding to the reflected ultrasound echoes; and
an external processing unit operationally connected to said ultrasound probe for controlling said ultrasound probe and receiving the data from said ultrasound probe.

26. The ultrasound imaging system according to claim 25 wherein said external processing unit is connected to said ultrasound probe via a control connection.

27. The ultrasound imaging system according to claim 26 wherein said control connection is wired.

28. The ultrasound imaging system according to claim 26 wherein said control connection is wireless.

29. The ultrasound imaging system to claim 26 wherein said non-transitory local storage medium is connected to said receiving unit independent of said control connection.

30. The ultrasound imaging system according to claim 25 wherein said non-transitory local storage medium is removably placed inside said ultrasound probe.

31. The ultrasound imaging system according to claim 25 wherein said non-transitory local storage medium is located near said ultrasound probe.

32. The ultrasound imaging system according to claim 25 wherein said ultrasound probe further comprises a displaying unit for displaying 2D images based upon the reflected ultrasound echoes.

33. The ultrasound imaging system according to claim 25 wherein said ultrasound probe further comprises a displaying unit for displaying 3D images based upon the reflected ultrasound echoes.

34. The ultrasound imaging system according to claim 25 wherein said ultrasound probe further comprises a displaying unit for displaying 2D images based upon the reflected ultrasound echoes before said non-transitory local storage medium stores the data for 3D/4D images based upon the reflected ultrasound echoes.

35. The ultrasound imaging system according to claim 25 wherein said ultrasound probe is a hand-held device.

36. The ultrasound imaging system according to claim 25 wherein said ultrasound probe further comprises a displaying unit for displaying 3D/4D images based upon the reflected ultrasound echoes before said non-transitory local storage medium stores the data for 2D images based upon the reflected ultrasound echoes.
